(19) United States
(12) Patent Application Publication
(10) Pub. No.: US 2020/0181664 A1
(43) Pub. Date: Jun. 11, 2020

(54) METHODS AND SYSTEMS FOR CONVERSION OF BIOMASS MATERIALS INTO BIOFUELS AND BIOCHEMICALS

(71) Applicant: ExxonMobil Research and Engineering Company, Annandale, NJ (US)

(72) Inventors: Arsam Behkish, Flemington, NJ (US); C. Rebecca Locker, Annandale, NJ (US); Giovanni Pilloni, Jersey City, NJ (US); Kelsey M. McNeely, Lambertville, NJ (US); Changyub Paek, Bridgewater, NJ (US); Ashish B. Mhadeshwar, Garnet Valley, PA (US); Eric B. Shen, Great Falls, VA (US)

(21) Appl. No.: 16/707,804

(22) Filed: Dec. 9, 2019

Related U.S. Application Data
(60) Provisional application No. 62/777,411, filed on Dec. 10, 2018.

(51) Int. Cl. C12P 19/14; C02F 11/12; C12P 7/10; C02F 3/34; C13K 1/02; C12P 19/02

(52) U.S. Cl. CPC C12P 19/14; C02F 11/12; C12P 7/10; C02F 3/342; C12P 2203/00; C12P 19/02; C12Y 302/01004; C02F 2303/06; C12P 2201/00; C13K 1/02

(57) ABSTRACT

The present disclosure relates to methods and systems for converting biomass into biofuels and biochemicals. In particular, the present disclosure relates to methods and systems for converting biomass comprising lignocellulosic material into biofuels and biochemicals, such as those comprising fatty acid esters, that contribute to reduction of greenhouse gas emissions.

### CROSS-REFERENCE TO RELATED APPLICATION

This application claims priority to U.S. Provisional Application No. 62/777,411, filed Dec. 10, 2018, which is herein incorporated by reference in its entirety.

### FIELD

The present disclosure relates to methods and systems for converting biomass into biofuels and biochemicals and, in particular, to methods and systems for converting biomass comprising lignocellulosic material into biofuels and biochemicals that contribute to reduction of greenhouse gas emissions.

### BACKGROUND

Global efforts are underway for the development of sustainable sources of energy, including biofuels and biochemicals, to reduce reliance on fossil fuels and reduce greenhouse gas emissions. For example, the United States, through the Energy Independence and Security Act (EISA) of 2007 and subsequently the expanded Renewable Fuel Standard (RFS) program, aims to increase the production of renewable fuel, particularly for use in transportation, by increasing amounts each year relative to petroleum-based fuels. Accordingly, the development of renewable or sustainable energy is of particular interest.

The use of biomass for making sustainable energy has been a focus for achieving reduced greenhouse gas emissions (GHG). The RFS targets are therefore set to reduce emissions based on feedstock source of the biofuel. Currently, such sustainable energy efforts have focused on so-called “first-generation” biofuels derived from food-related biomass and the fermentable sugars therein. For example, fermentable sugars have been derived from sugarcane, chemically transesterified vegetable oils, animal fats, and the like to produce bioethanol and biofuel. A primary source of food-related first-generation biomass currently used for producing sustainable energy is derived from corn (e.g., corn starch). However, such food-related biomasses, such as corn, are major global food sources and, accordingly, the first-generation efforts for producing sustainable energy competes with food availability. Furthermore, the reduction in GHG emissions associated with first-generation biofuels is dampened primarily due to the uncertainty associated with land use change.

As a result, new efforts, including mandates from the RFS program, have been focused on so-called “second-generation” biofuels derived from non-food biomass to meet the demand for sustainable energy without competing with food resources. These non-food biomass sources include, for example, residues from agriculture, forestry, and municipal solid waste. It is estimated that second-generation sustainable energy could satisfy a sizable percentage of the transportation fuel and diesel demands in the near future, thereby decreasing reliance on fossil fuels. Furthermore, the potential to reduce GHG emissions is greater for second-generation biofuels than for first-generation. However, the RFS classifications for Advanced Biofuels (up to 60% reduction in GHG emissions) and for Cellulosic Biofuels (greater than 60% reduction in GHG emissions) require each biofuel producer to submit evidence of the actual GHG emissions reduction by providing a life-cycle assessment (LCA) of the process. Not all second-generation biofuels will necessarily qualify as “Cellulosic Biofuel.” Currently, there is no metric that measures the efficiency of biorefineries (e.g., for lignocellulose material, as described below) as a function of GHG emissions reductions for the specific target of producing “Cellulosic Biofuel” or other biofuels.

A source of second-generation biomass that has been of interest is non-food biomass comprising lignocellulose material. Lignocellulose is present in all plant biomass, and is a complex of lignin, hemicellulose, and cellulose present in plant cell walls. Such lignocellulosic material, including those derived from agricultural waste, forest residue, and energy crops, is readily available and does not compete with food resources to supply fermentable sugars that can be harnessed to produced biofuels and biochemicals.

However, lignocellulosic material can be difficult to convert to useful products. For example, one current method utilizes pyrolysis to convert the lignocellulosic material to pyrolysis oil. The resulting pyrolysis oil can include a variety of compounds, including a high percentage of oxygenates and/or organic acids. But, due to the high percentage of oxygenates and organic acids, hydroprocessing of the pyrolysis oil into useful products can be costly and energy-intensive, thus reducing the benefit of converting the pyrolysis oil to fuel products. Further, pyrolysis oil can include a variety of compounds, such as ketones, aldehydes, and phenols, which interfere with the ability of alternative bacterial conversion into useful products.

### SUMMARY

The present disclosure relates to methods and systems for converting biomass into biofuels and biochemicals and, in particular, to methods and systems for converting biomass comprising lignocellulosic material into biofuels and biochemicals that contribute to reduction of greenhouse gas emissions across the product life-cycle.

In one or more aspects, the present disclosure provides a method for producing at least one or more biofuel and/or biochemical products with lower greenhouse gas emissions than conventionally produced product. The method comprises deconstructing a biomass comprising lignocellulosic material into hemicellulose, cellulose, lignin, and initial monosaccharides derived therefrom, thereby producing a deconstruction water effluent and a deconstructed biomass effluent. The deconstructed biomass effluent is enzymatically hydrolyzed to cause the cellulose to release additional monosaccharides, thereby forming a hydrolysate (sugar stream) effluent. Lignin is separated from the hydrolysate effluent to form a lignin-rich effluent and a liquid phase lignin-free effluent, where the liquid phase lignin-free effluent is further purified to form a purified effluent. The purified effluent is concentrated to increase its sugar concentration, resulting in an excess water effluent and a sugar concentrated effluent. Aerobic fermentation of the sugar concentrated effluent using microorganisms converts the sugars in the sugar concentrated effluent into fatty acids within the cells of the microorganisms. The fatty acids are transesterified into fatty acid esters using one or more alcohols, thereby producing at least one or more of a biofuel and/or a biochemical.

In one or more aspects, the present disclosure provides a system comprising a biorefinery that includes integrated equipment to convert a biomass to produce at least one or more of a biofuel and/or biochemical. The biorefinery is configured to perform a method for the production of at least one or more biofuel and/or biochemical. The method comprises deconstructing a biomass comprising lignocellulosic material into hemicellulose, cellulose, lignin, and initial monosaccharides derived therefrom, thereby producing a deconstruction water effluent and a deconstructed biomass effluent. The deconstructed biomass effluent is enzymatically hydrolyzed to cause the cellulose to release additional monosaccharides, thereby forming a hydrolysate effluent. Lignin is separated from the hydrolysate effluent to form a lignin-rich effluent and a liquid phase lignin-free effluent, where the liquid phase lignin-free effluent is further purified to form a purified effluent. The purified effluent is concentrated to increase its sugar concentration, resulting in an excess water effluent and a sugar concentrated effluent. Aerobic fermentation of the sugar concentrated effluent using microorganisms converts the sugars in the sugar concentrated effluent into fatty acids within the cells of the microorganisms. The fatty acids are transesterified into fatty acid esters using one or more alcohols, thereby producing at least one or more of the biofuel and/or a biochemical.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 is a schematic flowchart demonstrating one or more aspects of the biological conversion platform of sugars obtained from lignocellulosic biomass of the present disclosure.

FIG. 2 is a chart illustrating the relationship between potential scalability and greenhouse emission reduction compared to traditional fossil fuel source of the biological conversion platform of the present disclosure.

### DETAILED DESCRIPTION

The present disclosure relates to methods and systems for converting biomass into biofuels and biochemicals and, in particular, to methods and systems for converting biomass comprising lignocellulosic material into biofuels and biochemicals that contribute to reduction of greenhouse gas emissions.

Global demands are motivating the need for the development of sustainable energy, such as biofuels and biochemicals, which can decrease greenhouse gas emissions, decrease reliance on fossil fuels, and not compete with food sources. These demands are heightened by increasing environmental regulations for the decarbonization of energy. Lignocellulosic material (also referred to as lignocellulosic biomass) can be used to satisfy these demands. Lignocellulosic biomass is readily available and may be derived from agricultural residue and waste, forest residue, and energy crops.

An initial life cycle assessment of the use of sustainable energy derived from lignocellulosic biomass estimates an upward of 50% potential decrease in greenhouse gas emissions compared to diesel derived from fossil fuels. Indeed, current available collections of corn stover from corn production in the U.S. could satisfy 15% of the domestic distillate fuel demand. Accordingly, bioconversion processes that can convert lignocellulosic biomass, particularly at commercial scales, have the potential to greatly reduce greenhouse gas emissions without competing with important food resources.

The methods and systems described herein provide for a biological conversion platform of sugars obtained from lignocellulosic biomass using a genetically modified Escherichia coli (E. coli), a naturally occurring microorganism found in healthy human intestines. In particular, the methods and systems described herein provide for an E. coli biological conversion platform of lignocellulosic biomass, where such E. coli metabolism and physiology is optimized for the conversion of sugars into biofuels and biochemicals, such as fatty acid methyl ester (FAME). Unlike current processes, the methods and systems are commercially scalable, utilizing a direct bioconversion pathway of abundantly available lignocellulosic-derived sugars into distillate range molecules (e.g., FAME) and, in some instances, without the need for fossil fuel-based hydrotreatments. Moreover, the methods and systems derived herein can utilize a wide range of biomass material feeds for conversion of lignocellulosic compounds therein, and permit production of a wide range of distillate products, further enhancing commercial scalability.

In some embodiments, the present disclosure provides for a method and system that converts lignocellulosic material derived from a variety of biomass sources. As described in greater detail hereinbelow, the embodiments of the present disclosure include, first deconstructing non-food biomass comprising lignocellulosic material into its primary components of hemicellulose, cellulose, and lignin. The hemicellulose and cellulose are further hydrolyzed to release pentose and hexose monosaccharides. The lignin is separated from the hydrolyzed material and may be burnt in a steam generator (e.g., encompassing a boiler), for example, to produce energy, such as steam and/or electricity. Separately, the mixture of pentose and hexose monosaccharides is purified and concentrated, and thereafter fed into a bioreactor containing genetically modified E. coli bacteria for conversion into biofuels and/or biochemicals. The process of the present disclosure advantageously allows for feedstock flexibility (i.e., various types of readily available biomass may be used, as substantially all plant matter contains lignocellulosic material), direct production of biofuels and/or biochemicals, efficient use of resources and land, and significant reduction in greenhouse gas emissions as compared to fuels and chemicals derived from fossil fuels. Accordingly, the methods and systems described herein meet or exceed governmental regulations and greenhouse gas emissions targets (e.g., set by the EISA) without competing with biomass food sources. Further, as compared to current lignocellulosic biofuel processes, the embodiments described herein rely on aerobic fermentation using an integrated system flow that allows for closed-loop reuse of organic byproducts of the system (e.g., generating power from the combustion of lignin); permits obtention of product directly from the bioconversion step without additional product upgrading, such as hydrotreatment; does not require high energy separation steps, like distillation; and reduces the necessity for fossil fuel inputs during the biorefining. In addition, unlike current biodiesel that is produced from vegetable oil, the biofuel produced by process described herein uses lignocellulosic feedstock that does not directly compete with food production.

One or more illustrative embodiments incorporating the embodiments of the present disclosure are included and presented herein. Not all features of a physical implementation are necessarily described or shown in this application for the sake of clarity. It is understood that in the development of a physical embodiment incorporating the embodiments of the present disclosure, numerous implementation-specific decisions must be made to achieve the developer's goals, such as compliance with system-related, business-related, government-related, and other constraints, which vary by implementation and from time to time. While a developer's efforts might be time-consuming, such efforts would be, nevertheless, a routine undertaking for those of ordinary skill in the art and having benefit of this disclosure.

Unless otherwise indicated, all numbers expressing quantities of ingredients, properties such as physical properties, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present disclosure. At the very least, and not as an attempt to limit the application of the doctrine of equivalents to the scope of the claim, each numerical parameter should at least be construed in light of the number of reported significant digits and by applying ordinary rounding techniques. Where the term “less than about” or “more than about” is used herein, the quantity being modified includes said quantity, thereby encompassing values “equal to.” That is “less than about 3.5%” includes the value 3.5%, as used herein.

While compositions and methods are described herein in terms of “comprising” various components or steps, the compositions and methods can also “consist essentially of” or “consist of” the various components and steps.

Various terms as used herein are defined hereinbelow. To the extent a term used in a claim is not defined below, it should be given the broadest definition persons in the pertinent art have given that term as reflected in one or more printed publications or issued patents.

As used herein, the terms “lignocellulosic material,” “lignocellulosic biomass,” and “lignocellulose,” and grammatical variants thereof, are used herein interchangeably and are broad terms encompassing a complex of namely hemicellulose, cellulose, and lignin present in the cell walls of woody plants. “Lignocellulosic feedstock,” and grammatical variants thereof, includes biomass materials that comprise lignocellulosic material including, but not limited to, agricultural residues and waste (e.g., corn stover, wheat straw, bagasse), forest residues (e.g., woodchips), energy crops (e.g., switch grass, wheatgrass, bamboo), and the like, and any combination thereof.

As used herein, the term “biomass,” and grammatical variants thereof, refers to biological, organic matter that can be converted to fuel. The biomass for use in the embodiments of the present disclosure refers to organic plant matter.

As used herein, the term “biofuel,” and grammatical variants thereof, refers to a fuel derived directly or indirectly from biological, organic matter, used as an energy source (e.g., to produce heat or power).

As used herein, the term “biochemical,” and grammatical variants thereof, refers to a chemical product or intermediate derived directly or indirectly from a biological process.

As used herein, the term “biorefinery,” and grammatical variants thereof (e.g., “biorefining”), refers to a facility that integrates biomass conversion processes and equipment to produce biofuels and/or biochemicals from biomass. The equipment for use in the biorefinery systems of the present disclosure includes any suitable equipment for performing the methods described herein. Such equipment includes, but is not limited to, mechanical size-reduction equipment (e.g., grinders, millers, and the like), transport conduits (e.g., hoses, pipes, conveyers, and other fluid conduits), separation equipment (e.g., decanters, centrifuges, distillation columns, molecular sieves, and the like), non-reactor tanks, seed train tanks, bioreactors, water filtration equipment, filtration equipment (e.g., belt filters, vacuum belt filter, filter press, and the like), concentration equipment (e.g., evaporators, vapor-compression evaporation equipment, molecular sieves, and the like), steam generators, heat exchangers, pumps, compressors, boilers, water purification systems (e.g., reverse osmosis, filtration, deionizers, aerobic and anaerobic digesters, and the like), storage tanks, and the like, and any other equipment suitable for use in performing the methods described herein, and any combination thereof.

As used herein, the terms “transesterification” and “transesterified,” and grammatical variants thereof, refers to the process of exchanging an alkoxy group of an ester compound with another alcohol.

As used herein, the term “genetically modified,” and grammatical variants thereof, refers to a microorganism (e.g., a bacteria) containing genetic material that has been manipulated in a laboratory through techniques of genetic engineering to produce one or more desired characteristics.

The embodiments described herein comprise a series of concurrent, countercurrent, and single pass steps for the conversion of lignocellulosic material into biofuels and/or biochemicals. The first step in the biological conversion platform of the present disclosure includes deconstruction of a biomass comprising lignocellulosic material into its primary components of hemicellulose, cellulose, and lignin using one or more pretreatments. While certain specific pretreatments are described below, it is to be appreciated that any pretreatment known to one of skill in the art that achieves the desired outcome, as described below, may be employed in the embodiments of the present disclosure. Such pretreatments may include, but are not limited to, physical, physio-chemical, chemical, or biological methods. Examples include, but are not limited to, mechanical pretreatments (e.g., grinding, milling, and the like), hot water or steam (explosion) pretreatments, ammonia fibre expansion (AFEX) pretreatments, acid or alkaline pretreatments (e.g., sulfuric acid, sodium hydroxide, and the like), oxidizing agent pretreatments (e.g., hydrogen peroxide, employing ozonolysis, and the like), sulfite pulping, fungal pretreatments, enzymatic pretreatments, solvent-assisted extraction, and the like, and any combination thereof.

During pretreatment, hemicellulose releases pentose and hexose monosaccharide sugars. Pentose encompasses any monosaccharide comprising five atoms of carbon, such as xylose, rhamnose, arabinose, and the like. Hexose encompasses any monosaccharide comprising six atoms of carbon, such as glucose, galactose, mannose, and the like. The relative amounts of pentose and hexose sugars released from hemicellulose depends on the source of lignocellulosic biomass and the employed pretreatment technologies. The pretreatment technologies can be tuned by someone skilled in the art in order to release the maximum amount of each sugar component. Other by-products may additionally be released during hemicellulose pretreatment such as, for example, organic acids (e.g., formic acid, acetic acid, carboxylic acid, furoic acid, and the like), furans (e.g., furfurals, hydroxymethylfurfuals, and the like), and any combination thereof. Hemicellulose pretreatment may be achieved by any of the suitable methods described above, and in some embodiments, is achieved using an acid pretreatment (e.g., by employing a mild acid reaction using, for example, a dilute sulfuric acid solution). The acid pretreatment may be performed with loadings in the range of about 5 milligrams (mg) of acid per gram (g) of biomass to about 30 mg of acid per g of biomass, such as preferably about 10 mg of acid per g of biomass, encompassing any value and subset therebetween. The concentration of pentose and hexose, as well as the type and concentration of by-products may depend on a number of factors including, but not limited to, the type of biomass being pretreated, the pretreatment method selected, and the like, and any combination thereof.

Cellulose (as well as the remaining hexose oligomers from the hemicellulose) is thereafter treated to release hexose monosaccharides, which may be achieved by any suitable methods described above and, in some embodiments, is achieved using enzymatic hydrolysis. Enzymes derived from various sources may be used, including bacteria or fungi. The enzymes may be purchased commercially and/or manufactured directly at a biorefining site (i.e., at a location that the biological conversion of biomass containing lignocellulosic materials is performed, according to the embodiments described herein). Examples of suitable enzymes may include, but are not limited to, one or more cellulases and/or hemicellulases that aid in the decomposition of cellulose. Such cellulases may include, but are not limited to, endoglucanases, exoglucanases, β-glucosidase, and any combination thereof. Enzyme loadings can vary depending on the enzyme mix, but may be in the range of about 5 mg of enzyme per g of cellulose to about 25 mg of enzyme per g of cellulose, such as preferably about 10 mg of enzyme per g of cellulose, encompassing any value and subset therebetween.

After hydrolysis, according to the embodiments described herein, the effluent stream from the hydrolysis, generally referred to as “hydrolysate,” comprises various compounds represented primarily by water, a mixture of monosaccharides of pentose and hexose, lignin, and the various produced by-products. Some such by-products may be considered harmful, or otherwise toxic or inhibitors, to the biological conversion process, such as the microorganism used in the conversion process (e.g., a genetically modified E. coli ) to convert the monosaccharides into final desired biofuel or biochemical products. Accordingly, in some embodiments, the hydrolysate may be conditioned prior to the bioconversion step. Such conditioning is considered to be part of the pretreatment deconstruction portion of the biological conversion process.

In one or more embodiments, the step of conditioning the hydrolysate according to the methods and systems of the present disclosure may be a three-part conditioning step. In the first portion of the three-part conditioning step, lignin is removed from the hydrolysis effluent (i.e., the hydrolysate), which may be referred to as a separation stage. Lignin may be removed by any suitable methods including, but not limited to, lignin depolymerization, lignin filtration, and the like, and any combination thereof. In some embodiments, the insoluble lignin in the aqueous hydrolysate stream may be removed by any type of filtration (e.g., vacuum filtration). Once removed, the wet lignin can be dried and repurposed. For example, the dried lignin can be sold as a source of revenue, burned in a steam generator (e.g., a boiler) to generate steam for power production (e.g., to produce steam for a turbine to produce electricity), and the like, and any combination thereof. In some instances, the repurposed lignin may be used to supply heat and power for one or more aspects of the biological conversion process described herein.

The now lignin-free or substantially (i.e., mostly but not necessarily wholly) lignin-free stream in the biological conversion process typically has a relatively low concentration of monosaccharides (i.e., sugar) of typically in the range of about 5% to about 20%, or about 10% to about 15%, by weight of the lignin-free stream, encompassing any value and subset therebetween. Although not necessary, in some embodiments, the lignin-free stream is further concentrated to facilitate production of biofuel and/or biochemicals, such as to reduce equipment size requirements (e.g., smaller bioreactors, and the like) and facilitate processing. Accordingly, when used, in the second portion of the three-part conditioning step, the concentration step is performed in order to enhance the sugar concentration in the range of about 40% to about 50%, or about 45% to about 50%, by weight of the lignin-free stream, encompassing any value and subset therebetween. Sugar concentration may be achieved by any suitable methods including, but not limited to, vapor-compression evaporation (e.g., mechanical vapor compression or thermocompression), membrane filtration (e.g., ultrafiltration membranes), reverse osmosis, and the like, and any combination thereof.

During the concentration step of the lignin-free effluent, other harmful by-products (e.g., the toxins/inhibitors described above) may also be concentrated, which can effectively render the microorganism used later in the biological conversion process ineffective. Accordingly, the third part of the three-part conditioning step includes a purification step (or clean-up step) to remove by-products and other undesirable impurities. Purification may be achieved by any suitable methods including, but not limited to, physical, chemical, or biochemical purification procedures, such as the use of chemical reducing agents, adsorption beds (e.g., ion exchange resin beds, to activated carbon beds, and the like), liquid-liquid extraction, evaporation, bio-conversion (e.g., use of enzymes), and the like, and any combination thereof.

After completion of the conditioning step, the concentrated and purified hydrolysate is fed into an aerobic bioreactor comprising a genetically modified bacteria, such as E. coli, that is initially grown (e.g., replicated in a series of batch reactors of a seed train) and used to metabolically convert the sugars into fatty acid. The fatty acid is thereafter transesterified using one or more alcohols including, but not limited to, methanol, ethanol, and the like, and any combination thereof. For example, the fatty acid may be transesterified using methanol to produce a fatty acid methyl ester (FAME) or ethanol to produce a fatty acid ethyl ester (FAEE). The effluent therefrom is sent to a recovery block where the organic hydrocarbon is removed from the aqueous stream using a series of decanter vessels and further purified, such as to a purification level of 99% by weight (wt %) or higher (e.g., 99.5 wt %), with the balance being aqueous phase with impurities. Such purification may be achieved by any suitable methods including, but not limited to, centrifugation, water washing, membranes, and the like, and any combination thereof.

Various waste waters are obtained throughout the biological conversion process, as described in greater detail hereinbelow, including from the pretreatment, hydrolysis, conditioning, and other processes. These waste waters can be reprocessed, such as by using an anaerobic and/or aerobic digester to remove organic impurities (or wastes) therefrom, including further coupling with water filtration methods. Biogas (i.e., gaseous fuel, such as methane, produced by fermentation of organic matter) and sludge (i.e., organic-rich slurry stream) generated in an anaerobic digester may be burned in a boiler to generate steam for turbines to produce power (e.g., steam turbine to generate electricity, and the like), which can be utilized in the biological conversion processes of the present disclosure or in other processes requiring such power. Filtered water from an aerobic digester can be recycled back into the biological conversion processes described herein, as well. Accordingly, the biological conversion processes of the present disclosure allow for heat, power, and steam integration.

Any suitable genetically modified microorganism may be used in accordance with the methods and systems described herein. In some embodiments, the microorganism is genetically modified using modern, available molecular biology tools to enhance the microorganism's metabolism and physiology to optimize the conversion of biomass monosaccharides into fatty acids, such as fatty acid esters (e.g., FAME or FAEE). An example of a suitable genetically modified microorganism for use in the embodiments described herein includes, but is not limited to, the genetically modified E. coli disclosed in U.S. Patent Publication No. 2017/0175152, the entirety of which is incorporated herein by reference. Fatty acid-derived biofuels and biochemicals are viable, renewable, sustainable, and cost-effective alternatives to traditional fossils. The pathway for fatty acid biosynthesis in bacteria, such as E. coli, generally is carried out by a type II fatty acid synthase (FAS) enzyme. FAS is a multi-enzyme protein that catalyzes fatty acid synthesis in the presence of an alcohol by one or more biological pathways, which can be harnessed to produce biofuels and/or biochemicals, as described herein.

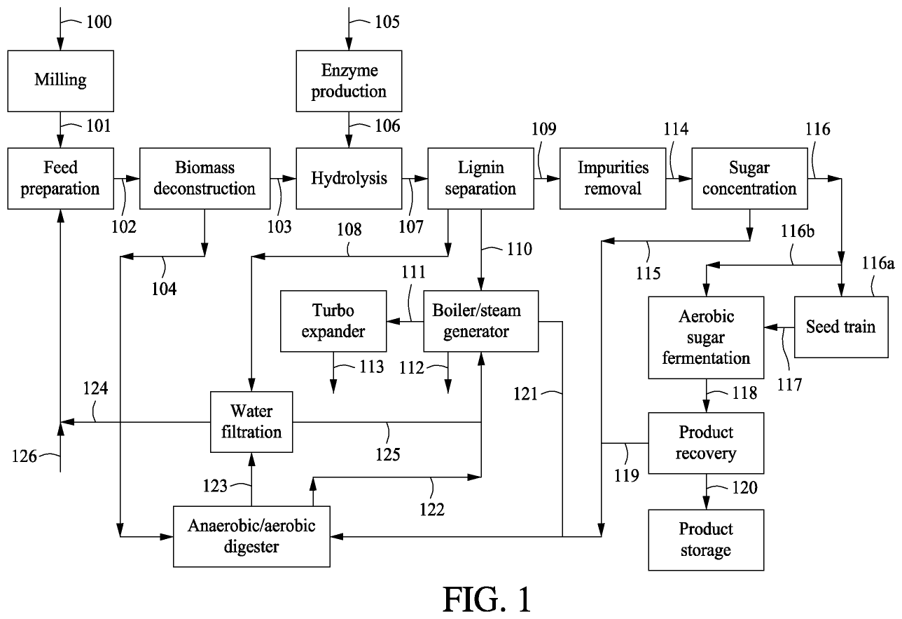

Referring now to FIG. 1, illustrated is a schematic flowchart demonstrating one or more aspects of the biological conversion platform of sugars obtained from lignocellulosic biomass of the present disclosure. As shown, in one or more embodiments, raw material biomass 100 comprising lignocellulosic material(s) is milled or cut into pieces at a biorefinery location to an appropriate, desired size. In some instances, such size may be in the range of about 1 centimeters (cm) to about 10 cm in length, such as preferably about 3 cm to about 7 cm in length, encompassing any value and subset therebetween, regardless of the shape of the milled material. As discussed previously, the biomass 100 may be one or more of a variety of plant matter, such as, for example, corn stover, switchgrass, or a combination thereof. The now-milled biomass 101 is transported for feed preparation. In some embodiments, the milled biomass 101 has a preferred moisture content for feed preparation, such as in the range of about 10% to about 40% moisture by weight of the milled biomass 101, such as preferably about 20% moisture by weight of the milled biomass 101, encompassing any value and subset therebetween. Depending, for example, on the moisture content of the milled biomass 101 (e.g., on the type of biomass, the storage conditions of the biomass, and the like), the biomass 101 may be slurried (i.e., made into a thin and viscous fluid).

In some embodiments, the milled biomass 101 may be slurried in an amount of from about 0% (i.e., need not be slurried) to about 30% by weight of the milled biomass 101, such as preferably about 20% to about 25% by weight of the milled biomass, encompassing any value and subset therebetween, thereby forming prepared feed 102. In some embodiments, the biomass 101 may be slurried with water. Alternatively, the now-milled biomass 101 may be conveyed directly as a solid feed such that the 101 stream is identical to the prepared feed 102 (i.e., did not have to be slurried).

The prepared feed 102, as shown in FIG. 1, is transported to the deconstruction portion of the biological conversion platform, also known as the pretreatment step. In this step, as described hereinabove, the prepared feed 102 is deconstructed into its main constituent parts—hemicellulose, cellulose, and lignin—using one or more pretreatment methods, such as an acid pretreatment coupled with a steam (explosion) pretreatment. During the pretreatment (deconstruction), hemicellulose may be further converted to pentose and hexose monosaccharides. In some embodiments, the yield of pentose monosaccharides from dry biomass feed may be in the range of about 10% to about 30%, such as about 20% to about 25% by weight of the biomass, encompassing any value and subset therebetween. In some embodiments, condensed water 104 (also referred to herein as “deconstruction water 104”) is removed from the deconstruction portion and may contain about 1 to 2 wt. % organic impurities and may be sent to an anaerobic and/or aerobic digester for treatment. The resultant water therefrom (e.g., 123 of FIG. 1) may thereafter be reused and/or filtered (e.g., water filtration portion in FIG. 1). In some embodiments, the deconstruction water 104 that has been treated and/or filtered may be reused within the biological conversion process shown in FIG. 1, and as described in greater detail below. Further, in some instances, the anaerobic digester may be used to produce and harness biogas (consisting mostly of methane) and an organic-rich, high-solid content (sludge).

The remaining deconstruction biomass effluent 103 (also referred to as pretreatment effluent) comprising hemicellulose, cellulose, lignin, and released sugars is thereafter treated in the hydrolysis portion of the biological conversion platform by enzymatic hydrolysis, such as by use of cellulose enzyme, to release the remaining sugars therefrom, particularly from the remaining cellulose, typically in a hydrolysis reactor. The enzymatic hydrolysis portion converts at least the cellulose to hexose monosaccharides, such as glucose, and may yield additional pentose monosaccharides (e.g., in addition to those obtained from the deconstruction of the hemicellulose). A typical yield of hexose monosaccharides from dry biomass feed may range between about 25% to about 45% by weight, such as about 30% to about 40% by weight, encompassing any value and subset therebetween.

As shown in FIG. 1, in some embodiments, the enzyme used for the hydrolysis portion is prepared on-site at the biorefinery location (block labeled “enzyme production”) and is fed 106 to the hydrolysis reactor for performance of the hydrolysis portion of the methods and systems described herein. One or more feeds, represented as feed 105, may be necessary for providing various nutrients (e.g., sugars, vitamins, salts, and the like) to support the production of the enzyme in the enzyme production block. Alternatively or in addition, the enzymes may be produced at another location or otherwise outside of the flow of the biological conversion process and thereafter fed 106 into the hydrolysis reactor, such as in any fermentation equipment, appropriate laboratory, or purchased commercially, without departing from the scope of the present disclosure.

Referring still to FIG. 1, after enzymatic hydrolysis, the resultant sugar stream effluent 107, also called hydrolysate effluent, is processed to remove lignin and insoluble solids in the lignin separation block. Lignin may be removed from the hydrolysate effluent 107 by any suitable methods, including those described hereinabove, such as by belt or vacuum belt filtration. The filtration process removes all or substantially all lignin and insoluble solids in the lignin-rich effluent 110, while retaining about 99% of the sugars in the liquid phase lignin-free effluent 109 (including substantially lignin-free). During such filtration processes, water 108 may be injected to facilitate filtration and removal of the lignin and insoluble solids. The water 108 may be obtained from the anaerobic/aerobic digestion process of waste water and subsequent water filtration of the digested water 123 thereof (the “water filtration block”).

The lignin-rich effluent 110 may comprise a moisture content, such as about 20% to about 40% moisture content, and can be transported to a boiler/steam generator, as shown in FIG. 1. The steam generator may be used to generate heat to produce high pressure, superheated steam, for example. In another embodiment, the moisture content in the lignin-rich effluent 110 can be lowered by drying and using heat integration or additional process fuel. The steam created by the steam generator may be used to satisfy the steam demands 112 of the biological conversion process, such as, for example, enzyme production, ion exchange bed regeneration (e.g., the “impurities removal” block), sterilization needs (e.g., sterilization of bioreactors including seed trains and aerobic fermentation), and the like, and any combination thereof. Excess high pressure superheated steam 111 may be sent to a turbo expander (or expansion turbine) equipped, for example, with an electrical generator to produce electricity which may thereafter be used to supply the electrical demands 113 of the biological conversion process. Excess power is exported to support other electrical needs within the biorefinery and/or outside of the biorefinery, as appropriate. Accordingly, the biological conversion platform further recycles resources to provide integrated steam and electrical needs.

The liquid phase lignin-free effluent 109 obtained from the lignin separation block (e.g., by belt filtration) may comprise various impurities, such as organic impurities that are removed (the “impurities removal” block). The method of removing the organic impurities may be any suitable methods, including those listed hereinabove, such as by use of a resin bed, a reactive extraction, or a bio-conversion of the impurities using microorganism strains, including genetically modified microorganism strains. The resultant, purified effluent 114 comprises a sugar concentration that is relatively low compared to the desired sugar concentration. Accordingly, the purified effluent 114 is concentrated in the sugar concentration block. Typically, the purified effluent 114 comprises a sugar content in the range of about 5% to about 20% by weight of the purified effluent 114. In the sugar concentration portion of the biological conversion process described herein, the sugar content is concentrated to about 30% to about 60% by weight of the sugar concentrated effluent 116 (effluent after the sugar concentration portion), or preferably to about 40% to about 50% by weight of the concentrated effluent 116, encompassing any value and subset therebetween. Any suitable sugar concentration method may be used, as described hereinabove, such as mechanical vapor compression. Excess water effluent 115 may be removed from the sugar concentration portion and sent to the anaerobic and/or aerobic digester (e.g., separately or through the save avenue (e.g., pipe or tube) from the deconstruction water 104) for further processing and reuse in the biological conversion process.

With continued reference to FIG. 1, the concentrated effluent 116 is split into two or more streams (two shown in FIG. 1) for inoculation of a seed train and one or more production (or fermentation) bioreactors, respectively. Seed trains utilize multiple (at least two) bioreactors in series operating in batch mode, which may be progressively larger, for generating an adequate number of microorganism cells (e.g., volume) to inoculate a production bioreactor. For example, as shown in FIG. 1, a first concentrated effluent stream 116 is used to inoculate the seed train, which may be in the range of about 1% to about 10% by weight of the original concentrated effluent 116, or preferably about 3% to about 8% by weight of the original concentrated effluent 116, encompassing any value and subset therebetween. The seed train portion of the biological conversion process described herein may be an aerobic batch process in which an initial seed of genetically modified microorganisms, such as the genetically modified E. coli described above is inoculated with the first concentrated effluent stream 116a. That is, the concentrated sugar in the first concentrated effluent stream 116a is used to provide nutrients and initiate bacterial culture growth (replication) in the seed train block, into which additional nutrients may also be provided during the seed train portion. In some embodiments, the aerobic batch seed train block utilizes a series of progressively larger bioreactors to achieve a desired amount of microorganism growth. Upon achieving the desired amount of microorganism growth, the resultant seed train effluent 117 (i.e., the culture media and microorganisms from the seed train block) is used to seed a production, or main fermentation, bioreactor (the “aerobic sugar fermentation block”). Although not shown, the aerobic sugar fermentation block may comprise one or more production (or fermentation) bioreactors, without departing from the scope of the present disclosure.

The balance of the concentrated effluent 116, second concentrated effluent stream 116b, is used to as a direct feed to a production bioreactor along with the seed train effluent 117, and thereafter to facilitate conversion of sugars to fatty acids, as described hereinabove. The second concentrated effluent stream 116b will accordingly serve as initial nutrients for bacterial growth (replication) of the seed train effluent 117 contents, into which additional nutrients may be provided during the aerobic sugar fermentation block and, thereafter, as nutrients for fermentation of sugars to fatty acids.

First, an initial portion of the second effluent stream 116b is combined with the seed train effluent 117 and introduced to the production reactor. The combined second effluent stream 116b and seed train effluent 117 is introduced into the production reactor at an initial volume in the range of about 10% to about 40% by volume of the bioreactor, or preferably about 20% to about 30% by volume of the bioreactor, encompassing any value and subset therebetween. Accordingly, the initial portion of the second effluent stream 116b is included in the production bioreactor to achieve the desired concentration in combination with the seed train effluent 117. This initial portion of the second effluent stream 116b is used to provide nutrients to continue the growth (replication) of the microorganisms in the seed train effluent 117.

The microorganism population in the production bioreactor is monitored until the population concentration reaches a desired or optimum level; generally, the concentration is optimum when the microorganisms begin producing (excreting) fatty acid. Upon reaching the desired level, the balance of the second concentrated effluent stream 116b is introduced into the production reactor to facilitate the fermentation of monosaccharide sugars into fatty acids in the cells of the genetically modified microorganisms as described hereinabove. The fermentation and biological conversion process of the embodiments described herein is aerobic, with the microorganisms producing fatty acids within their cells and in the process releasing carbon dioxide (CO₂). Oxygen (air) is pumped into the bioreactor to maintain aerobic respiration in the bioreactor, and in some embodiments, is sparged into the production bioreactor, such as from the bottom thereof. The CO₂ may be vented at any concentration, such as at about 19% concentration and the balance is air used in the aerobic process.

During fermentation of the fatty acids (or conversion of the sugars to fatty acids) by the microorganisms, alcohol is fed (e.g., to the reactor) for transesterification of fatty acids. As described hereinabove, the alcohol may be methanol or ethanol, for example, to produce FAME or FAEE, respectively. Methanol may be obtained commercially and introduced into the production reactor. Ethanol may also be obtained commercially; alternatively, or in addition, ethanol may be produced in situ in the production reactor through fermentation of sugar(s) therein. It is to be appreciated that a combination of alcohols (e.g., a combination of methanol and ethanol) may be used in the production reactor, resulting in a combination of fermented fatty acids. Typically, the concentration of alcohol included in the production reactor may be an amount in the range of about 1% to about 10% by weight of the sugar concentrated effluent described herein (i.e., the hydrolysate effluent fed to the reactor), or preferably about 3% to about 7% by weight of the sugar concentrated effluent, encompassing any value and subset therebetween. The alcohol may be injected into the one or more production reactors by any suitable means, such as at the bottom thereof, the top thereof, any other one or more injection points along the axis of the production bioreactor, and the like, and any combination thereof.

As stated above, the aerobic sugar fermentation block may comprise one or more production bioreactors. Such production fermentation reactors may operate in batch, fed-batch, or continuous mode. The one or more production bioreactors may be any suitable fermentation bioreactors including, but not limited to, those using sparged stirred-tank bioreactors, bubble column bioreactors, and the like, and any combination thereof. Heat generated during the aerobic sugar fermentation block may be removed from the one or more production bioreactors by any suitable methods including, but not limited to, use of internal cooling lines, jacketed walls, loop around (external) cooling lines, external refrigeration, and the like, and any combination thereof. In some embodiments of the present disclosure, the one or more production bioreactors comprise internal cooling lines in combination with stirred tanks that are designed as a coil around the mixing impeller shaft to act as a draft tube to enhance liquid (and cooling) circulation. Moreover, one or more impellers of different types may be employed in the one or more production bioreactors, such as axial impeller(s), radial impeller(s), and the like, and any combination thereof. Accordingly, in some embodiments, greater than one and/or greater than one type of impeller may be used in the production bioreactors described herein, without departing from the present disclosure. In some embodiments, the one or more production bioreactors may include a bubble column with internal cooling lines.

After conversion of the microorganism fatty acids into fatty acid esters with the alcohol in the one or more production bioreactors, the bioreactor effluent 118 is transported to a production recovery portion of the biological conversion process described herein. At the product recovery block in the process, as shown in FIG. 1, a two or more (e.g., a series) of decanter vessels may be used to first separate the resultant aqueous phase effluent 119 and organic phase effluent 120. The aqueous phase effluent 119 may be transported to the anaerobic and/or aerobic digester to for further processing and reuse in the biological conversion process. That is, the anaerobic and/or aerobic digesters are configured in the biological conversion platform process of the present disclosure to receive any or all, preferably all, of the waste waters (e.g., deconstruction water 104 (from pretreatment), excess water effluent 115, aqueous phase effluent 119, and the like) generated during the process, including the condensed steam 121 from the steam turbine generator. It is to be appreciated that the waste water lines from the various sources of waste water may be shared along any length thereof, including the entirety of the length, without departing from the scope of the present disclosure. The digested water 123 may be further filtered (the “water filtration” block” and reused in the biological conversion process of the present disclosure, as described herein, such as to the filter press for lignin separation (stream 108), the feed preparation step (stream 124), and/or the steam generator. Various filtration methods may be used in the water filtration block of the present disclosure including, but not limited to, ultrafiltration, nanofiltration, reverse osmosis, and the like, and any combination thereof. In this manner, as well as in other ways, the biological conversion platform of the present disclosure, including that shown in FIG. 1, facilitates an integrated process that recycles certain elements for conservation of power and resources. In some embodiments, additional fresh water 126 may also be introduced into the biological conversion processes of the present disclosure, as needed. In other embodiments, additional fresh water (not shown) and/or recycled water may be introduced into the system to facilitate enzyme production, enzymatic hydrolysis, product recovery (e.g., when a scrubber column is used), utility needs, and the like, and any combination thereof.

As provided above, the anaerobic digester may produce biogas, such as biogas stream 122, as shown in FIG. 1. This biogas stream 122 may be transported to the boiler/steam generator to increase steam generation. Further contributing to the integrated nature of the biological conversion processes described herein, an aerobic digester may be used to further decompose remaining organic species carried over from the anaerobic digester.

The bioreactor effluent 118 may be decanted to separate the aqueous phase and organic phase thereof and, in some embodiments, the organic phase may be further processed to increase the purity of the resultant biofuel and/or biochemical, such as by centrifugation, water washing, and the like, and any combination thereof, to form a purified organic phase effluent 120. The purified organic phase effluent 120 may be transported to product storage for use as a biofuel and/or a biochemical

The biological conversion process(es) described herein and with reference to FIG. 1 is specifically configured to optimize the production of biofuels and biochemicals with the classification of “Cellulosic Biofuel” which demonstrate greater than 60% reduction in greenhouse gas emission relative to traditional fossil fuel hydrocarbon sources. Accordingly, the scalability of a biorefinery to produce such biofuels and/or biochemicals, as described herein, represents an important metric for determining the potential magnitude of GHG emissions reduction. A metric for assessing the reduction of GHG emissions is shown in Formula 1, where Ψ defines the scalability efficiency of the biorefineries.

Ψ=(Biorefinery Capacity)^{Carbon %} Formula 1

According to Formula 1, a biorefinery capacity is related to the size of the biorefinery itself relative to the maximum feedstock processing capability and, for use herein, “Carbon %” is the carbon efficiency of the biological conversion platform process(es) of the present disclosure (number of carbon in the final bio-product per number of carbon in the biomass feed). Thus, the value of Ψ is an indication of the efficiency of the biorefinery as it is scaled up (i.e., as biorefinery is able to process greater amounts of biomass into greater amounts of biofuels and/or biochemicals).

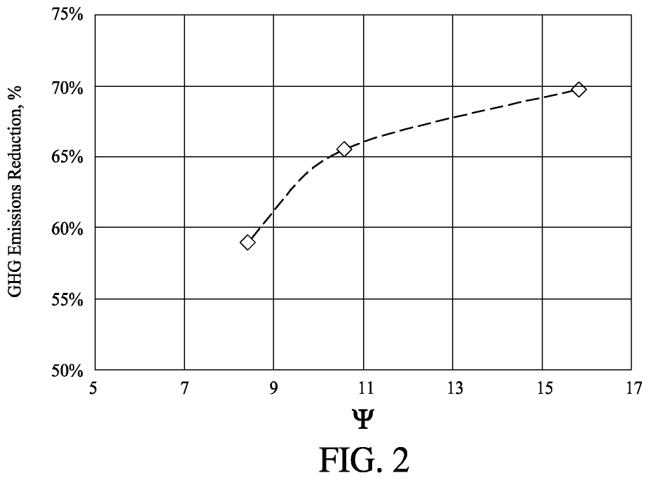

Referring now to FIG. 2, illustrated is an assessment of the biological conversion platform described herein according to one or more embodiments to demonstrate the relationship between potential scalability and GHG emissions reduction compared to traditional fossil fuel source. Three different scenarios were evaluated with varying biorefinery capacities and carbon efficiencies. Biorefinery capacities ranged from production rates of about 2,500-10,000 barrels of biofuel per day, and carbon efficiencies of the biological conversion of sugar to fuel varied from about 85%-90% yield on sugar monomers. As shown in FIG. 2, as Ψ increases, the reduction in GHG increases. Indeed, even at the lowest Ψ value, a biorefinery employing the embodiments of the present disclosure would experience a decrease in GHG emissions of 59% compared to traditional fossil fuel sources, which according to RFS qualifies as an “Advanced Biofuel.” As Ψ increases, moreover, the GHG emissions reduction is estimated at greater than 60%, which can qualify according to RFS as “Cellulosic Biofuel.” Accordingly, the biological conversion platform methods and systems described herein increase both process efficiency, as well as economy of scale and can guide the optimum design of a biorefinery with the highest GHG emissions reductions.

Embodiments disclosed herein include:

Embodiment A: A method comprising: deconstructing a biomass comprising lignocellulosic material into hemicellulose, cellulose, lignin, and initial monosaccharides derived therefrom, thereby producing a deconstruction water effluent and a deconstruction biomass effluent; enzymatically hydrolyzing the deconstruction biomass effluent to cause the cellulose to release additional monosaccharides, thereby forming a hydrolysate effluent; separating lignin from the hydrolysate effluent, thereby forming a lignin-rich effluent and a liquid phase lignin-free effluent; purifying the liquid phase lignin-free effluent, thereby forming a purified effluent; concentrating the purified effluent to increase a sugar concentration thereof, thereby forming an excess water effluent and a sugar concentrated effluent; aerobically fermenting the sugar concentrated effluent using microorganisms in the presence of oxygen to produce fatty acids; and transesterifying the produced fatty acids into fatty acid esters using an alcohol, thereby producing at least one or more of a biofuel and biochemical.

Embodiment B: A system comprising: a biorefinery that comprises integrated equipment to convert biomass to produce at least one or more of a biofuel and biochemical, the biorefinery configured performing the method of: deconstructing a biomass comprising lignocellulosic material into hemicellulose, cellulose, lignin, and initial monosaccharides derived therefrom, thereby producing a deconstruction water effluent and a deconstruction biomass effluent; enzymatically hydrolyzing the deconstruction biomass effluent to cause the cellulose to release additional monosaccharides, thereby forming a hydrolysate effluent; separating lignin from the hydrolysate effluent, thereby forming a lignin-rich effluent and a liquid phase lignin-free effluent; purifying the liquid phase lignin-free effluent, thereby forming a purified effluent; concentrating the purified effluent to increase a sugar concentration thereof, thereby forming an excess water effluent and a sugar concentrated effluent; aerobically fermenting the sugar concentrated effluent using microorganisms in the presence of oxygen to produce fatty acids; and transesterifying the produced fatty acids into fatty acid esters using an alcohol, thereby producing the at least one or more of the biofuel and biochemical.

Embodiments A and B may have one or more of the following additional elements in any combination:

Element 1: Wherein prior to the deconstructing, the biomass is mechanically reduced in size by milling.

Element 2: Wherein the deconstructing is achieved using a pretreatment selected from the group consisting of an acid pretreatment, a steam (explosion) pretreatment, and any combination thereof.

Element 3: Wherein the enzymatically hydrolyzing is achieved using a cellulase enzyme.

Element 4: Wherein the purifying is achieved using belt filtration.

Element 5: Wherein the concentrating is achieved using vapor-compression evaporation.

Element 6: Wherein microorganisms are genetically modified E. coli.

Element 7: Wherein the aerobically fermenting is initially performed in a seed train.

Element 8: Wherein the alcohol is selected from the group consisting of methanol, ethanol, and any combination thereof.

Element 9: Further comprising transporting one or more of the deconstruction water and the excess water effluent to one or both of an anaerobic digester and an aerobic digester to remove organic impurities therefrom.

Element 10: Further comprising transporting one or more of the deconstruction water and the excess water effluent at least to an anaerobic digester to remove organic impurities therefrom, wherein an anaerobic output of the anaerobic digester is one or more of biogas and sludge, and further comprising transporting one or more of the biogas and sludge to a steam generator to generate power.

Element 11: Further comprising transporting one or more of the deconstruction water and the excess water effluent at least to an aerobic digester to remove organic impurities therefrom, and wherein an aerobic output of the aerobic digester is digested water, and further comprising filtering the digested water.

Element 12: Further comprising transporting one or more of the deconstruction water and the excess water effluent at least to an anaerobic digester to remove organic impurities therefrom,

By way of non-limiting example, exemplary combinations applicable to A and B include: 1 and 2; 1 and 3; 1 and 4; 1 and 5; 1 and 6; 1 and 7; 1 and 8; 1 and 9; 1 and 10; 1 and 11; 1 and 12; 2 and 3; 2 and 4; 2 and 5; 2 and 6; 2 and 7; 2 and 8; 2 and 9; 2 and 10; 2 and 11; 2 and 12; 3 and 4; 3 and 5; 3 and 6; 3 and 7; 3 and 8; 3 and 9; 3 and 10; 3 and 11; 3 and 12; 4 and 5; 4 and 6; 4 and 7; 4 and 8; 4 and 9; 4 and 10; 2 and 11; 4 and 12; 5 and 6; 5 and 7; 5 and 8; 5 and 9; 5 land 10; 5 and 11; 5 and 12; 6 and 7; 6 and 8; 6 and 9; 6 and 10; 6 and 11; 6 and 12; 7 and 8; 7 and 9; 7 and 10; 7 and 11; 7 and 12; 8 and 9; 8 and 10; 8 and 11; 8 and 12; 9 and 10; 9 and 11; 9 and 12; 10 and 11; 10 and 12; 11 and 12; and any non-limiting combination of one, more, or all of 1, 2, 3, 4, 5, 6, 7, 8, 9, 10, 11, and/or 12.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

1. A method comprising:
deconstructing a biomass comprising lignocellulosic material into hemicellulose, cellulose, lignin, and initial monosaccharides derived therefrom, thereby producing a deconstruction water effluent and a deconstruction biomass effluent;
enzymatically hydrolyzing the deconstruction biomass effluent to cause the cellulose to release additional monosaccharides, thereby forming a hydrolysate effluent;
separating lignin from the hydrolysate effluent, thereby forming a lignin-rich effluent and a liquid phase lignin-free effluent;
purifying the liquid phase lignin-free effluent, thereby forming a purified effluent;
concentrating the purified effluent to increase a sugar concentration thereof, thereby forming an excess water effluent and a sugar concentrated effluent;
aerobically fermenting the sugar concentrated effluent using microorganisms in the presence of oxygen to produce fatty acids; and
transesterifying the produced fatty acids into fatty acid esters using an alcohol, thereby producing at least one or more of a biofuel and biochemical.

2. The method according to claim 1, wherein prior to the deconstructing, the biomass is mechanically reduced in size by milling.

3. The method according to claim 1, wherein the deconstructing is achieved using a pretreatment selected from the group consisting of an acid pretreatment, a steam (explosion) pretreatment, and any combination thereof.

4. The method according to claim 1, wherein the enzymatically hydrolyzing is achieved using a cellulase enzyme.

5. The method according to claim 1, wherein the purifying is achieved using belt filtration.

6. The method according to claim 1, wherein the concentrating is achieved using vapor-compression evaporation.

7. The method according to claim 1, wherein microorganisms are genetically modified E. coli.

8. The method according to claim 1, wherein the aerobically fermenting is initially performed in a seed train.

9. The method according to claim 1, wherein the alcohol is selected from the group consisting of methanol, ethanol, and any combination thereof.

10. The method according to claim 1, further comprising transporting one or more of the deconstruction water and the excess water effluent to one or both of an anaerobic digester and an aerobic digester to remove organic impurities therefrom.

11. The method according to claim 10, wherein an anaerobic output of the anaerobic digester is one or more of biogas and sludge.

12. The method according to claim 11, further comprising transporting one or more of the biogas and sludge to a steam generator to generate power.

13. The method according to claim 10, wherein an aerobic output of the aerobic digester is digested water, and further comprising filtering the digested water.

14. A system comprising:
a biorefinery that comprises integrated equipment to convert biomass to produce at least one or more of a biofuel and biochemical, the biorefinery configured performing the method of:
deconstructing a biomass comprising lignocellulosic material into hemicellulose, cellulose, lignin, and initial monosaccharides derived therefrom, thereby producing a deconstruction water effluent and a deconstruction biomass effluent;
enzymatically hydrolyzing the deconstruction biomass effluent to cause the cellulose to release additional monosaccharides, thereby forming a hydrolysate effluent;
separating lignin from the hydrolysate effluent, thereby forming a lignin-rich effluent and a liquid phase lignin-free effluent;
purifying the liquid phase lignin-free effluent, thereby forming a purified effluent;
concentrating the purified effluent to increase a sugar concentration thereof, thereby forming an excess water effluent and a sugar concentrated effluent;
aerobically fermenting the sugar concentrated effluent using microorganisms in the presence of oxygen to produce fatty acids; and
transesterifying the produced fatty acids into fatty acid esters using an alcohol, thereby producing the at least one or more of the biofuel and biochemical.

15. The system according to claim 14, wherein prior to the deconstructing, the biomass is mechanically reduced in size by milling.

16. The system according to claim 14, wherein the deconstructing is achieved using a pretreatment selected from the group consisting of an acid pretreatment, a steam (explosion) pretreatment, and any combination thereof.

17. The system according to claim 14, wherein the enzymatically hydrolyzing is achieved using a cellulase enzyme.

18. The system according to claim 14, wherein the purifying is achieved using belt filtration.

19. The system according to claim 14, wherein the concentrating is achieved using vapor-compression evaporation.

20. The system according to claim 14, wherein microorganisms are genetically modified E. coli.

21. The system according to claim 14, wherein the aerobically fermenting is initially performed in a seed train.

22. The system according to claim 14, wherein the alcohol is selected from the group consisting of methanol, ethanol, and any combination thereof.

23. The system according to claim 14, further comprising transporting one or more of the deconstruction water and the excess water effluent to one or both of an anaerobic digester and an aerobic digester to remove organic impurities therefrom.

24. The system according to claim 23, wherein an anaerobic output of the anaerobic digester is one or more of biogas and sludge.

25. The system according to claim 24, further comprising transporting one or more of the biogas and sludge to a steam generator to generate power.

26. The system according to claim 23, wherein an aerobic output of the aerobic digester is digested water, and further comprising filtering the digested water.
